(19) United States
(12) Patent Application Publication
(10) Pub. No.: US 2005/0177151 A1
(43) Pub. Date: Aug. 11, 2005

(54) Irrigation sheath

(76) Inventors: Thomas P. Coen, Pleasanton, CA (US); Katie L. Krueger, San Jose, CA (US); Dorin Panescu, San Jose, CA (US); David K. Swanson, Campbell, CA (US); Miriam H. Taimisto, San Jose, CA (US)

Correspondence Address: Bingham McCutchen LLP;Suite 1800, Three Embarcadero Center, San Francisco, CA, 94111-4067, US

(21) Appl. No.: 10/625,194

(22) Filed: Jul. 23, 2003

Related U.S. Application Data
(62) Division of application No. 09/886,754, filed on Jun. 20, 2001, now abandoned.

(51) Int. Cl. A61B 18/18

(52) U.S. Cl. 606/41

(57) ABSTRACT

A medical system for performing a tissue ablation procedure comprises a guide sheath and an ablation catheter disposed within an internal lumen of the catheter. The guide sheath has a distal end that includes irrigation exit ports that are configured to perfuse irrigation fluid in a distal direction over the ablation electrode of the catheter when the distal end of the catheter protrudes from the guide sheath. In this manner, the ablation electrode can be advantageously cooled during the tissue ablation process, thereby maximizing the size and depth of the ablation lesion and reducing the duration of the ablation process.

### TECHNICAL FIELD

The present invention generally relates to medical devices, and more specifically, to methods and apparatus for cooling an ablation electrode during a therapeutic tissue ablation procedure.

### BACKGROUND OF THE INVENTION

For many years, catheters have had widespread application in the medical field. For example, mapping and ablation catheters have been extensively used in the treatment of cardiac arrhythmia. Cardiac arrhythmia treatments help restore the normal operation of the heart in pumping blood to the body. Mapping and ablation catheters play a critical role in these highly delicate treatments.

Typically, the catheters used in mapping and ablation procedures are steerable electrophysiological (“EP”) catheters that may be precisely positioned anywhere in the heart. These catheters are generally used during two distinct phases of treatment for heart arrhythmia. In one phase of treatment, the catheters are used to map the heart by locating damaged tissue cells. This involves locating damaged cells by steering the catheter to selected locations throughout the heart and detecting irregularities in the propagation of electrical wave impulses during contraction of the heart (a procedure commonly referred to as “mapping”). During the other phase of treatment, the same catheter is typically used to create thermal lesions at the location where damaged cells have been found (a procedure commonly referred to as “ablation”).

Ablation procedures using catheters are typically performed using radio frequency (“RF”) energy. In this regard, an EP catheter has one or more ablation electrodes located at its distal end. The physician directs energy from the electrode through myocardial tissue either to an indifferent electrode, such as a large electrode placed on the chest of the patient (in a uni-polar electrode arrangement), or to an adjacent electrode (in a bipolar electrode arrangement) to ablate the tissue. Once a certain temperature has been attained, resistance heating of the tissue located adjacent the one or more electrodes occurs, producing lesions at the targeted tissue.

Generally, ablation procedures require careful control of the amount of RF energy channeled to the catheter electrodes. When excessive thermal energy is applied to a catheter electrode during ablation procedures, blood protein and other biological tissue may coagulate on the electrode, creating an embolic hazard. Such build up of coagulant on the electrode also hinders the transmission of RF energy from the electrode into the target tissue, thereby reducing the effectiveness of the ablation procedure. Ideally, RF energy would be focused entirely on the targeted heart tissue without damaging the surrounding tissue or blood cells. That is, it would be highly preferable to be able to generate a relatively large lesion at a specifically defined area without altering, damaging, or destroying other surrounding tissue or blood.

In addition, it is generally desirable to be able to minimize the time it takes to complete an ablation procedure. Typically, the longer it takes to complete an ablation procedure, the greater the health risk to the patient. Also, the longer it takes to complete each ablation procedure, the higher the cost of treatment. The time required to perform an ablation procedure is related to how much thermal energy is directed towards the targeted tissue. That is, the greater the thermal energy directed towards the targeted tissue, the quicker the procedure can be performed. The amount of thermal energy that may be applied to the targeted tissue, however, is limited by damage that could potentially occur to the surrounding blood cells and tissue at high thermal energy levels. For the above reasons, an EP catheter that is able to efficiently dissipate excess heat would be highly desirable.

One suggested approach is to cool the electrode by pumping cooling fluid through the catheter, where it is recirculated to internally cool the catheter tip, or perfused out exit holes to externally cool the catheter tip. Although this approach provides a means of delivering heat-dissipating irrigation fluids to the tip region, it has certain drawbacks. For example, catheters, such as ablation catheters, are typically very small in size. The provision of a fluid flow path to the tip of a catheter occupies critical space within the catheter, thus limiting the incorporation of other valuable components, such as heat sensors, into the catheter. Further, designing and building catheters that can accommodate irrigation fluids may be costly and difficult, and may not always be effective in cooling the electrode tip region. Therefore, a system that can efficiently dissipate excess heat at the tip region of a catheter, without the need for substantially changing the design of the catheter, would be highly desirable.

### SUMMARY OF THE INVENTION

The present invention provides an irrigated sheath system and method for delivering fluids through a guide sheath. In this case of an ablation catheter, the fluid can be a room temperature or cooled irrigation fluid used to cool the ablation electrode of the catheter during a tissue ablation process.

In accordance with a first aspect of the present invention, a medical guide sheath for use with catheters comprises an internal lumen configured for housing a catheter. The sheath further includes an open distal end that comprises one or more fluid exit ports. The fluid exit ports are configured to advantageously perfuse fluid in a substantially distal direction over the catheter distal end when the catheter distal end protrudes from the open sheath distal end. For example, if the catheter is an ablation catheter with a distally mounted ablation electrode, room temperature or cooled irrigation fluid can be pumped over the ablation electrode during the ablation process. The guide sheath can be either steerable or fixed.

In accordance with a second aspect of the present inventions, the afore-described guide sheath and catheter can be combined, along with an irrigation fluid system, to form an irrigated medical system. In this regard, the irrigation fluid system is in fluid communication with the one or more fluid exit ports. The irrigation fluid system can supply various fluids to the guide sheath, including irrigation fluid, drugs, such as heparin, and contrast fluid for diagnostic procedures.

In accordance with a third aspect of the present inventions, a medical guide sheath comprises an elongated sheath body having an open distal end, an internal lumen formed within the sheath body, and a plurality of skives formed on an inner surface of the open distal end. The skives are in fluid communication with the internal lumen. In the preferred embodiment, the open distal end comprises a wall having a distally facing surface, and the plurality of skives extends proximally from the distally facing surface. The sheath may further comprise a proximally mounted fluid entry port that is in fluid communication with the internal lumen. Thus, pressurized fluid applied to the fluid entry port is conveyed through the internal lumen, through the skives, and out of the distal end of the guide sheath.

In accordance with a fourth aspect of the present inventions, a medical guide sheath comprises an elongated sheath body having an open distal end, an internal lumen formed within the sheath body, and a plurality of fluid exit ports located on the outer surface of the open distal end. The fluid exit ports extend through the wall of the open distal end in fluid communication with the internal lumen. Preferably, the outer surface of the open distal end comprises a plurality of skives that extends distally from the plurality of exit ports. The sheath may further comprise a proximally mounted fluid entry port that is in fluid communication with the internal lumen. Thus, pressurized fluid applied to the fluid entry port is conveyed through the internal lumen, out through the fluid exit ports, through the skives, and out of the distal end of the guide sheath.

In accordance with a fifth aspect of the present inventions, a medical guide sheath comprises an elongated sheath body having an open distal end, an internal lumen formed within the sheath body, a plurality of fluid lumens axially disposed within the wall of the open distal end, and a plurality of fluid exit ports located on the distally facing edge of the open distal end in fluid communication with the plurality of fluid lumens. The plurality of axially disposed fluid lumen can either be in fluid communication with the internal lumen, or extend the length of the sheath. The sheath may further comprise a proximally mounted fluid entry port in fluid communication with the axial fluid lumens. Thus, pressurized fluid applied to the fluid entry port is conveyed through the fluid lumens and out through the fluid exit ports. If the fluid lumens are in fluid communication with the internal lumen, the pressurized fluid is conveyed through the internal lumen prior to entering the fluid lumens.

### BRIEF DESCRIPTION OF THE DRAWINGS

FIG. 1 is a perspective view of a fixed irrigated sheath system that embodies features of the present invention.

FIG. 2A is a perspective view of one configuration of the distal end of the sheath of FIG. 1, wherein irrigation fluid exits through an annular aperture between the distal end of the catheter and the distal end of the sheath.

FIG. 2B is an end view of the sheath distal end of FIG. 2A.

FIG. 2C is a dissected side view of the sheath distal end of FIG. 2A.

FIG. 3 is a dissected side view of a sheath and a catheter, particularly illustrating a catheter locking mechanism.

FIG. 4A is a perspective view of another configuration of the distal end of the sheath of FIG. 1, wherein irrigation fluid exits through skives formed on the inner surface of the sheath distal end.

FIG. 4B is an end view of the sheath distal end of FIG. 4A.

FIG. 5A is a perspective view of still another configuration of the distal end of the sheath of FIG. 1, wherein irrigation fluid exits through fluid exit ports formed on the other surface of the sheath distal end.

FIG. 5B is a cross-sectional view of the sheath distal end of FIG. 5A taken along the line 5B-5B.

FIG. 6A is a perspective view of yet another configuration of a distal end of the sheath of FIG. 1, wherein irrigation fluid exits through fluid lumens axially disposed in the wall of the sheath distal end.

FIG. 6B is the end view of the sheath distal end of FIG. 6A.

FIG. 6C is a dissected side view of the sheath distal end of FIG. 6A.

FIG. 7 is a perspective view of a steerable irrigated sheath system that embodies features of the present invention.

### DESCRIPTION OF THE PREFERRED EMBODIMENTS

The present invention provides for an irrigated sheath system that is capable of delivering an irrigation fluid to the tip of a medical catheter (e.g., an ablation/mapping catheter) in a more efficient manner. With respect to ablation catheters, the present sheath system provides an increased fluid flow to the ablation electrode, thereby providing many advantages. For example, the efficient fluid flow provides for larger, longer and deeper lesions during the ablation process, as compared to other prior art cooled ablation systems. This becomes more significant when treating atrial flutter, which requires deep lesions in the isthmus, or for treating ventricular tachycardia, which requires deep lesions in the ventricles. In comparison to other prior art cooled ablation systems, the incidences of tissue charring, coagulation on electrodes, and popping are reduced, thus making the ablation process more safe. The present sheath system also reduces the number of RF applications, the duration of the ablation procedure and fluoroscopy time, and requires less power/temperature to create a lesion similar in size to prior art cooled ablation systems. The present sheath system allows the ablation tip electrode on the catheter to be reduced in diameter and length, thereby increasing the accuracy of mapping, providing a better electrogram recording, and allowing the catheter to be more easily steered and maneuvered. The irrigation sheath of the present invention may be optionally used with catheters that provide other functions, such as ultrasound imaging, blood withdrawal, fluid injection, blood pressure monitoring, and the like.

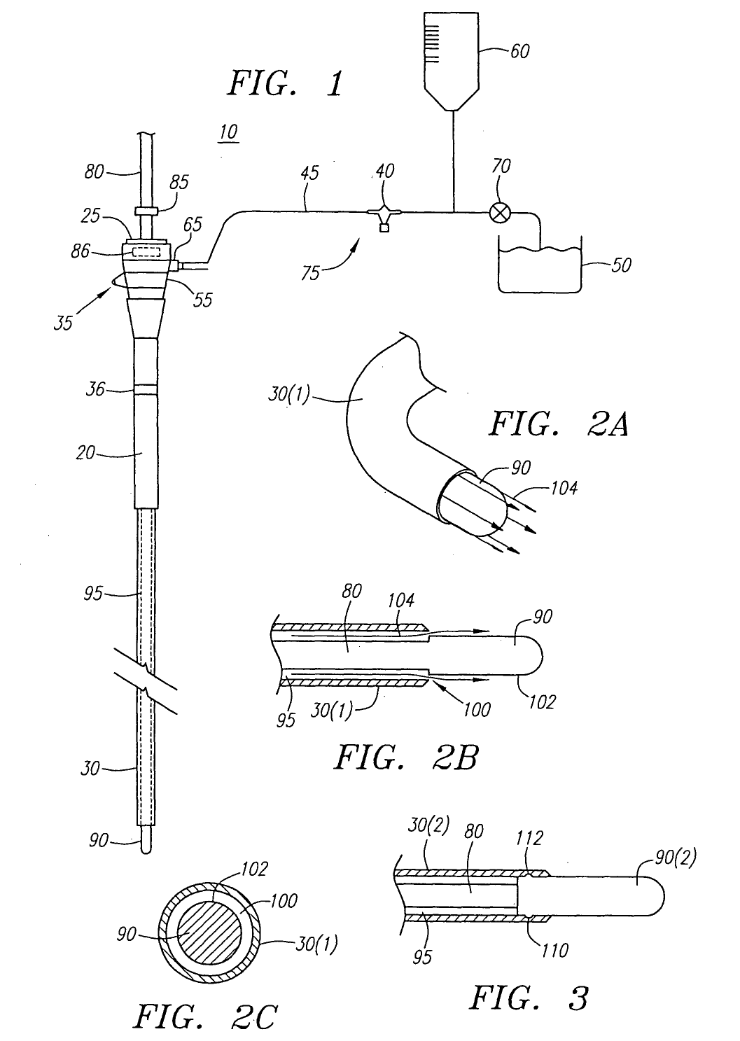

FIG. 1 shows a fixed sheath irrigation system 10 that can be used for irrigation during an ablation process. The system 10 includes an elongated fixed sheath 20 with a distal end 30 and a proximal end 35. The system 10 further includes a catheter 80 that is disposed within an internal fluid lumen 95 of the sheath 20. As will be discussed in further detail below, the fluid lumen 95 provides the system 10 with a means for conveying room temperature or cooled irrigation fluid from the sheath proximal end 35 to the sheath distal end 30. The catheter 80 includes a distally mounted ablation tip electrode 90 that can be controllably activated via an RF generator and controller (not shown) to therapeutically ablate surrounding tissue. The diameter of the ablation electrode 90 has a suitable size, e.g., 7F in diameter. During the ablation process, the ablation electrode 90 is preferably located partially outside or just distal to the sheath distal end 30, as illustrated in FIG. 1. It should be noted that, although the sheath distal end 30 is shown as having a pre-shaped rectilinear geometry, it can also have any pre-shaped curvilinear geometry that is adapted for specific applications, such as abnormalities in the right atrium or right inferior pulmonary vein. The sheath distal end 30 includes a radiopaque marker (not shown) to facilitate the location of the sheath distal end 30 with respect to the desired tissue area. The proximal end of the sheath 20 includes a remote anode ring 36 for unipolar recordings.

The fixed sheath 20 is made from a flexible, biologically compatible material, such as polyurethane or polyethylene, and has a suitable size, e.g., 7F. The sheath distal end 30 is preferably more flexible than the proximal end 35 to enhance the maneuverability of the sheath 20. To provide steerability to the sheath 20, an independent steering device, such as a steerable catheter, which may be the catheter 80 itself or a separate catheter, may optionally be used to control the movement of the sheath/catheter combination. An example of a steerable catheter used in ablation procedures is described in U.S. Pat. No. 5,871,525.

A hemostasis valve 55 is mounted on the proximal end 35 of the sheath 20, and includes a catheter port 25 for insertion of the catheter 80 into the fluid lumen 95 of the sheath 20. As will be discussed in further detail below, the system 10 includes a catheter locking mechanism. In particular, the proximal end of the catheter 80 includes an annular ridge 85, and the hemostasis valve 55 includes an annular indentation 86 located on the inside of the catheter port 25. Thus, as the catheter 80 is distally advanced through the fluid lumen 95 of the sheath 20, the annular ridge 85 engages the annular indentation 86, creating an interference fit therebetween and locking the catheter 80 in place relative to the sheath 20. In this regard, proper axial positioning of the ablation electrode 90 relative to the sheath distal end 30 is facilitated, the significance of which will be described in further detail below. Furthermore, the locked system 10 obviates the need for the physician to use both hands when maneuvering the sheath 20 and catheter 80. Alternatively, a reference mark can be located on a portion of the proximal end of the catheter 80 that, when aligned with the opening of the catheter port 25, indicates that the ablation electrode is properly located relative to the sheath distal end 30. The hemostasis valve 55 further includes a fluid entry port 65, which is in fluid communication with the fluid lumen 95.

The system 10 further includes a fluid feed system 75 for delivery of various fluids to the fluid lumen 95 of the sheath 20. Specifically, an intravenous bag 60 and a fluid reservoir 50 are in fluid communication with a fluid line 45, which is in turn in fluid communication with the fluid entry port 65 located on the hemostasis valve 55. The intravenous bag 60 contains a medical therapeutic or diagnostic fluid, such as heparin, drugs, or contrast fluid, which continuously flows under gravitational pressure through the fluid line 45 and sheath 20. The fluid reservoir 50 contains a room temperature or cooled irrigation fluid, such as saline, which is conveyed under pressure through the fluid line 45 via a pump 70. Alternatively, irrigation fluid can be provided to the fluid line 45 by a gravity feed, such as an intravenous bag, or a pressurized bag feed. A stopcock 40 controls the flow of fluid from the intravenous bag 60 and fluid reservoir 50 into the fluid line 45. Thus, a medical fluid and the irrigation fluid can be simultaneously conveyed through the fluid line 45, through the fluid lumen 95, and out the sheath distal end 30. Alternatively, the intravenous bag 60 and pump 70 can be connected directly to the stopcock 40, so that medical fluid and the irrigation fluid can be independently delivered to the sheath distal end 30. More alternatively, the hemostasis valve may include two fluid entry ports in fluid communication with the fluid lumen 95, in which case the intravenous bag 60 and pump 70 may be connected separately to the respective entry ports through two respective stopcocks to allow independent delivery of the medical fluid and irrigation fluid to the sheath distal end 30.

When fluid is pumped through the fluid lumen 95 of the sheath 20, it exits the distal end 30 and flows over the exterior surface of the ablation electrode 90. During an ablation procedure, this fluid takes the form of an irrigation fluid, which cools the ablation electrode 90, thereby facilitating the ablation process. This irrigation fluid may be, e.g., a 0.9% saline solution, which exhibits three times the electrical conductivity of blood and ten times the electrical conductivity of the myocardium of the heart. These characteristics aid in reducing the ohmic heat generated at the ablation electrode 90, thus eliminating, or at least reducing, the afore-mentioned problems with conventional ablation catheters.

The distal end 30 of the sheath 20 is configured, such that the irrigation fluid exits the distal end 30 in a distal direction over the ablation electrode 90. Referring to FIGS. 2A, 2B and 2C, a sheath distal end 30(1) is configured, such that an annular aperture 100 is formed between the fluid lumen 95 of the sheath 20 and an outer surface 102 of the ablation electrode 90 when the ablation electrode 90 partially protrudes out the distal end 30(1) and the irrigation fluid is pumped through the fluid lumen 95. In the illustrated embodiment, the section of the fluid lumen 95 located adjacent to the sheath distal end 30(1) has a diameter, such that the sheath distal end 30(1) loosely fits around the ablation electrode 90. In this case, the elastic characteristics of the sheath distal end 30(1) allows it to naturally expand in the presence of the pressurized irrigation fluid, thereby forming the annular aperture 100 between the sheath distal end 30(1) and the ablation electrode 90.

Alternatively, the section of the fluid lumen 95 at the sheath distal end 30(1) has a diameter that is slightly greater than the outer diameter of the ablation electrode 90 (e.g., 0.008 inch greater), in which case, the sheath distal end 30(1) need only minimally expand to form the annular aperture 100. It should be noted that, although in the illustrated embodiment, the annular aperture 100 is formed between the fluid lumen 95 of the sheath 20 and the outer surface 102 of the ablation electrode 90, the annular aperture 100 can alternatively be formed between the fluid lumen 95 of the sheath 20 and the outer surface of the catheter just proximal to the ablation electrode 90. In this case, the ablation electrode 90 should not be deployed so far from the annular aperture 100 that the cooling effects of the exiting irrigation fluid are not too substantially reduced.

In any event, the annular aperture 100 should be configured to maximize the percentage of the exterior surface of the ablation electrode 90 over which the irrigation fluid flows. A suitable dimension of the annular aperture 100 may be 0.004 inches per side. Thus, as can be seen from FIGS. 2A and 2B, the irrigation fluid generally follows flow path 104, i.e., it flows through the fluid lumen 95, exits out the annular aperture 100, and flows over the ablation electrode 90. It should be noted that it is desirable that the wall thickness of the sheath distal end 30(1) be as small as possible to facilitate flush contact between the partially protruding ablation electrode 90 and the tissue during parallel tissue ablations, i.e., when the longitudinal axis of the ablation electrode 90 is parallel to the surface of the ablated tissue.

As previously described, a proximal locking mechanism can be employed to ensure proper axial orientation of the ablation electrode 90 relative to the sheath distal end 30(1). Alternatively, the ablation electrode can be distally locked in place relative to the sheath 20. For example, in FIG. 3, an ablation electrode 90(2) and a sheath distal end 30(2) can be constructed with a ridge and indentation arrangement. In this configuration, an annular ridge 110 is formed on the ablation electrode 90(2), and a corresponding annular indentation 112 is formed on the inside wall of the sheath distal end 30(2). As the catheter 80 is distally advanced through the fluid lumen 95 of the sheath 20, the annular ridge 110 engages the annular indentation 112, creating an interference fit therebetween and locking the catheter 80 in place relative to the sheath 20.

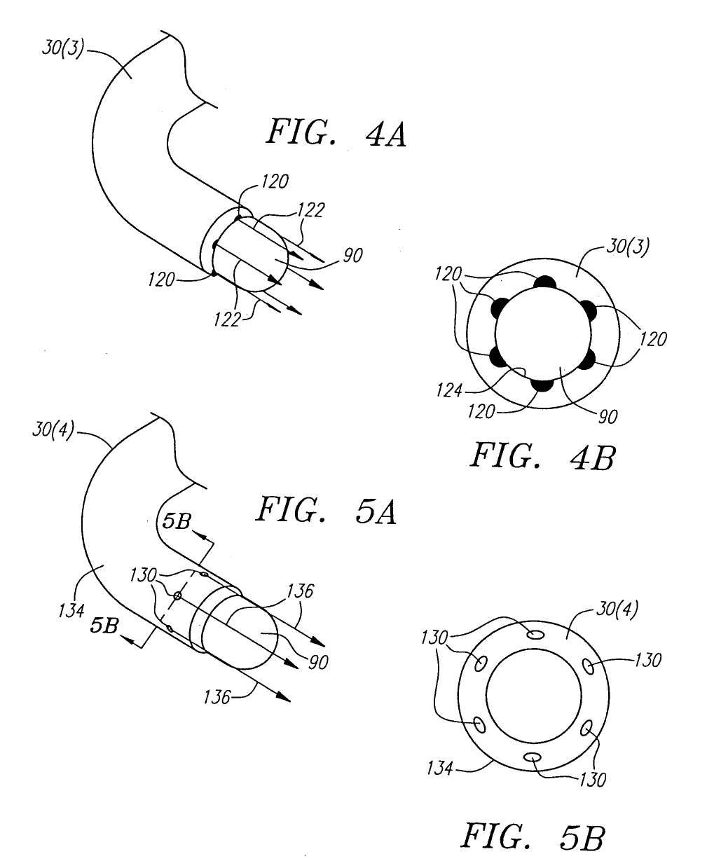

Referring to FIGS. 4A and 4B, an inner surface 124 of the sheath distal end 30(3) includes a plurality of skives 120. The skives 120 are in fluid communication with the fluid lumen 95 of the sheath 20, and the sheath distal end 30(3) is tightly fitted around the ablation electrode 90, forming a seal between the inner surface 124 of the sheath distal end 30(3) and the outer surface of the ablation electrode 90. Thus, when irrigation fluid is pumped through the fluid lumen 95 (shown in FIG. 2) and the ablation electrode 90 partially protrudes out the sheath distal end 30(3), the irrigation fluid exits the skives 120 and flows over the exterior surface of the ablation electrode 90. In the illustrated embodiment, the skives 120 extend the entire length of the sheath 20, resulting in a flow of irrigation fluid that is substantially isolated within the skives 120 along the length of the fluid lumen 95. Alternatively, the skives 120 extend only in the sheath distal end 30(3). In this case, the sheath 20 is loosely fitted around the catheter 80 proximal to the skives 120, resulting in an annular flow of irrigation fluid within the fluid lumen 95 that is then channeled into the skives 120 at the sheath distal end 30(3). In any event, the irrigation fluid exits the skives 120, flowing over the exterior surface of the ablation electrode 90, as shown by flow paths 122.

Referring now to FIGS. 5A and 5B, a distal end 30(4) of the sheath 20 includes fluid exit ports 130 located on an outer surface 134 of the sheath distal end 30(4). The exit ports 130 are disposed at a distally facing oblique angle to the longitudinal axis of the sheath 20, such that irrigation fluid flowing through the fluid lumen 95 exits the ports 130 in a distal direction and over the ablation electrode 90, as illustrated by flow path 136. To further enhance the cooling effects of the ablation electrode 90, this embodiment optionally includes skives on the inner surface of the distal end 30(4), as described with respect to FIGS. 4A and 4B.

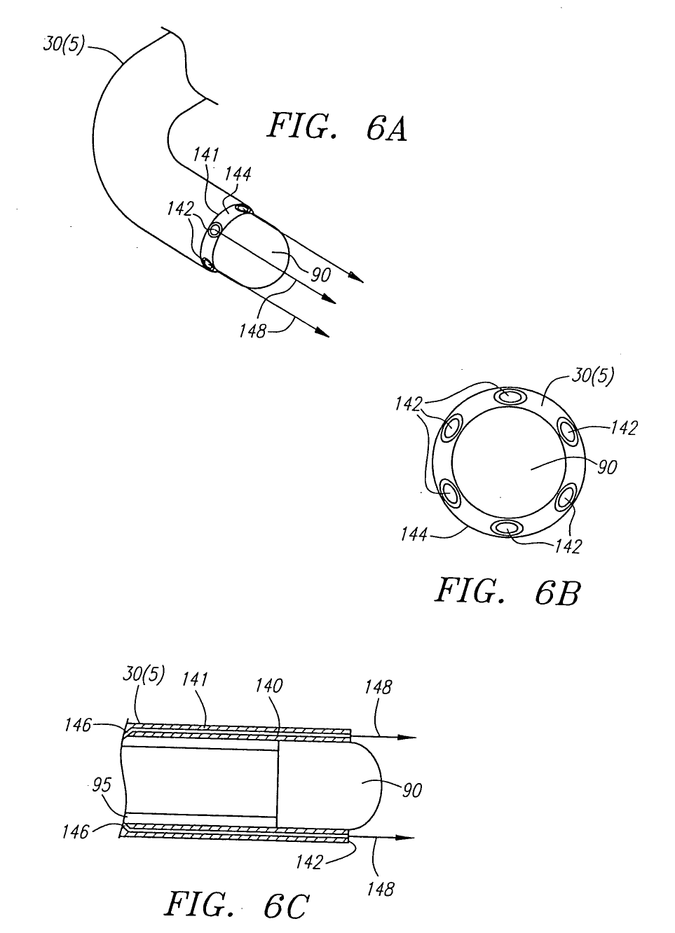

Referring to FIGS. 6A, 6B and 6C, a distal end 30(5) of the sheath 20 includes a plurality of fluid lumens 140 extending through a wall 141 of the distal end 30(5), terminating at fluid exit ports 142 located at a distal edge surface 144 of the sheath distal end 30(5). In the illustrated embodiment, the fluid lumens 140 are in fluid communication with the internal fluid lumen 95 via connecting channels 146 that extend partially through the wall 141 of the sheath distal end 30(5). Thus, irrigation fluid, pumped through the fluid lumen 95, flows through the connecting channels 146 into the fluid lumens 140, and out through the exit ports 142, where it flows over the exterior surface of the ablation electrode 90, as illustrated by flow path 148. Alternatively, the fluid lumens 140 extend the length of the sheath 20. In this case, the fluid lumens 140 are in direct fluid communication with the fluid entry port 65 located on the hemostasis valve 55 (shown in FIG. 1), in which case, the internal fluid lumen 95 can be used to transport other fluids. If the fluid lumens 140 do extend the length of the sheath 20, specific fluid lumens 140 can optionally be connected to different fluid sources such that, for example, one fluid lumen 140 may be used for irrigation fluids, while another can be used for drugs and/or flushing.

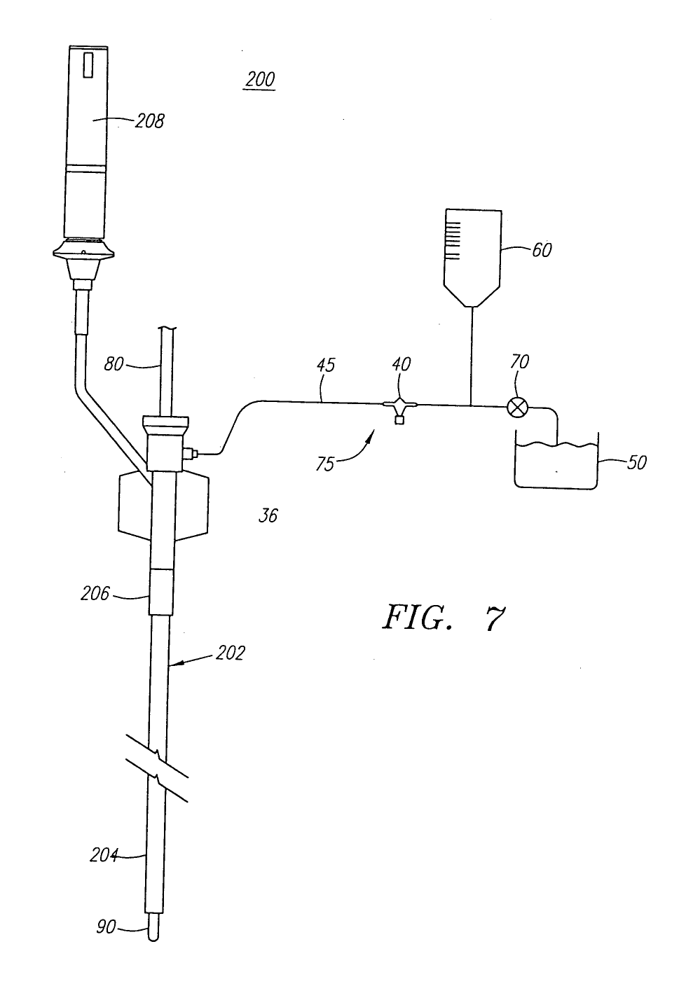

Referring now to FIG. 7, a steerable sheath irrigation system 200 is shown. It should be noted that, to the extent that the system 200 and system 10 described above use common features, identical reference numbers have been used. The system 200 differs from the system 10 in that it includes a steerable sheath 202, rather than a fixed sheath. The system 200 includes the aforementioned catheter 80, which may optionally be steerable as well. The steerable sheath 202 includes a distal end 204 and a proximal end 206. Attached to the proximal end 206 is a sheath handle 208, housing components for controlling and steering the steerable sheath 202. As with the system 10, the sheath distal end 204 can be configured in a number of ways to provide irrigation fluid to the ablation electrode 90 of the catheter 80. For example, the sheath distal end 204 can be configured in the manner described with respect to FIGS. 2-6.

Although particular embodiments of the present inventions have been shown and described, it will be understood that it is not intended to limit the invention to the preferred embodiments, and it will be obvious to those skilled in the art that various changes and modifications may be made without departing from the spirit and scope of the present invention. Thus, the invention is intended to cover alternatives, modifications, and equivalents, which may be included within the spirit and scope of the invention as defined by the claims. All publications, patents, and patent applications cited herein are hereby expressly incorporated by reference in their entirety for all purposes.

1-35. (canceled)

36. A medical system, comprising:
an elongated flexible catheter comprising a catheter distal end; and
an elongated flexible sheath comprising an open sheath distal end, an internal lumen configured to house said catheter, one or more open channels formed in an inner surface of said sheath distal end in fluid communication with said internal lumen, and one or more fluid exit ports located on said sheath distal end in fluid communication with said one or more open channels, wherein said one or more fluid exit ports are configured to perfuse fluid in a substantially distal direction over said catheter distal end when said catheter distal end protrudes from said open sheath distal end.

37. The medical system of claim 36, wherein said inner surface of said sheath distal end substantially forms a seal with an outer surface of said catheter distal end.

38. The medical system of claim 36, wherein said one or more fluid exit ports comprise a plurality of fluid exit ports.

39. The medical system of claim 36, wherein said catheter is an ablation catheter having a distally mounted ablation electrode.

40. The medical system of claim 36, further comprising a catheter locking mechanism configured for axially fixing said catheter relative to said sheath.

41. The medical system of claim 40, wherein said catheter locking mechanism comprises an annular ridge located on one of said catheter and said sheath, and an annular indentation located on the other of said catheter and said sheath, said annular ridge and said annular indentation configured for engaging each other when said catheter is advanced through said internal lumen of said sheath.

42. The medical system of claim 36, further comprising an irrigation fluid system in fluid communication with said internal lumen.

43. The medical system of claim 42, wherein said irrigation fluid system comprises a source of irrigation fluid and a pump for conveying said irrigation fluid under pressure to said one or more fluid exit ports.

44. The medical system of claim 42, wherein said irrigation fluid system comprises a source of another fluid that can be conveyed under pressure to said one or more fluid exit ports.

45. The medical system of claim 42, wherein said source of irrigation fluid is a source of cooled irrigation fluid.

46. The medical system of claim 36, wherein a proximal end of said sheath comprises a hemostasis valve.

47. The medical system of claim 36, wherein said sheath distal end is steerable.

48. The medical system of claim 36, wherein said sheath is an intravascular sheath, and said catheter is an intravascular catheter.

49. A medical guide sheath for use with an elongated flexible catheter, comprising:
an elongated flexible sheath body having an open distal end;
an internal lumen formed within said sheath body and being configured for housing the catheter;
one or more open channels formed in located on an inner surface of said sheath distal end in fluid communication with said internal lumen.

50. The medical guide sheath of claim 49, further comprising one or more fluid exit ports in fluid communication with said one or more open channels, said one or more fluid exits ports configured to perfuse fluid in a substantially distal direction.

51. The medical guide sheath of claim 49, wherein said one or more fluid exit ports comprises a plurality of fluid exit ports.

52. The medical guide sheath of claim 49, further comprising a hemostasis valve mounted on a proximal end of said sheath body.

53. The medical guide sheath of claim 49, wherein said open distal end is steerable.

54. The medical guide sheath of claim 49, further comprising one or more fluid exit ports located on said sheath distal end in fluid communication with said one or more open channels, wherein said one or more fluid exit ports are configured to perfuse fluid in a substantially distal direction.

55. The medical guide sheath of claim 49, wherein said guide sheath is an intravascular sheath.
